(19) United States
(12) Plant Patent Application Publication
(10) Pub. No.: US 2008/0313782 P1
(43) Pub. Date: Dec. 18, 2008

(54) Murtilla plant named 'Red Pearl - INIA'

(76) Inventors: Ivette Seguel Benitez, Temuco (CL); Adolfo Montenegro Barriga, Temuco (CL)

Correspondence Address: STRATTON BALLEW, 213 S 12TH AVE, YAKIMA, WA, 98902, US

(21) Appl. No.: 12/077,728

(22) Filed: Mar. 19, 2008

(30) Foreign Application Priority Data
Mar. 19, 2007 (CL) 731

(51) Int. Cl. A01H 5/00

(52) U.S. Cl. PLT/156

(57) ABSTRACT

‘Red Pearl—INIA’ is a new variety of murtilla plant, characterized by intense red berries having excellent flavor, appearance, and shipping and storage qualities.

### PRIORITY CLAIM

This application claims priority under 35 U.S.C. §119(f) of Chilean Plant Variety Rights Application Ser. No. 731, filed Mar. 19, 2007.

### CROSS-REFERENCE TO RELATED APPLICATIONS

None

### STATEMENT REGARDING FEDERALLY SPONSORED RESEARCH OR DEVELOPMENT

None

### GENUS AND SPECIES

Ugni molinae Turcz. (syn. Myrtus ugni)

### VARIETY DENOMINATION

Red Pearl—INIA

### BACKGROUND OF THE INVENTION

The murtilla plant, also known as “Chilean guava” or “u\ddot{n}i,” is native to Southern Chile. Fruit of the murtilla plant is used for fresh consumption, as well as for processing into jellies, syrups, desserts, and liqueurs. Until recently, commercially harvested murtilla fruit was obtained from wild murtilla plants, as there were no known, cultivated murtilla plants having the specific characteristics desireable for commercial production.

In 1996, the Instituto de Investigaciones Agropecuarias (Agricultural Research Institute) of Chile (INIA—Chile) initiated a research project to domesticate murtilla and develop selections suitable for commercial production. The research project involved the creation of a murtilla gene bank, the first such collection in Chile, as well as the study of suitable reproduction techniques; phenotypic characterization of the germplasm collection; evaluation of the chemical characteristics of the fruit; molecular characterization of the fruit; reproductive studies; agronomic evaluations; development of agronomic management guides; identification and development of new varieties; and economic and commercial studies. As a result of the murtilla research project, several new varieties of murtilla plant were identified by INIA—Chile and selected for advancement and commercialization.

‘Red Pearl—INIA’ is a new variety of murtilla plant developed by INIA—Chile through its murtilla research project. In 1996, germplasm samples were obtained from 103 murtilla plants at 36 collection sites in southern Chile. These samples were then cultivated at an INIA—Chile testing facility for observation and testing as described above. After extensive evaluation and successive asexual propagations, ‘Red Pearl—INIA’ was identified by the inventors as having characteristics that distinguish it from other murtilla plants, and make it particularly suitable for commercial production.

‘Red Pearl—INIA’ is distinguishable from wild murtilla and from its sister plant (‘South Pearl—INIA’, application filed concurrently herewith). Table 1 sets forth some of the distinguishing characteristics of ‘Red Pearl—INIA.’

TABLE 1
Comparison of ‘Red Pearl-INIA’ to ‘South Pearl-INIA’
and to wild murtilla

| | Red Pearl-INIA | South Pearl-INIA | Wild murtilla (avg.) |
|---|---|---|---|
| Skin Color | Light Red | Intense dark | Red |
| | | red | |
| Fruit Size | 1.0 | 1.0 | 1.0 to 1.5 cm |
| Harvest Yield | 0.9 kg/plant | 1.1 kg/plant | 0.6 kg/plant |

### BRIEF SUMMARY OF THE INVENTION

‘Red Pearl—INIA’ is a new variety of murtilla plant, characterized by intense dark red berries having excellent flavor, appearance, and shipping and storage qualities.

### BRIEF DESCRIPTION OF THE PHOTOGRAPHS

FIG. 1 shows the ‘Red Pearl—INIA’ murtilla plant; and

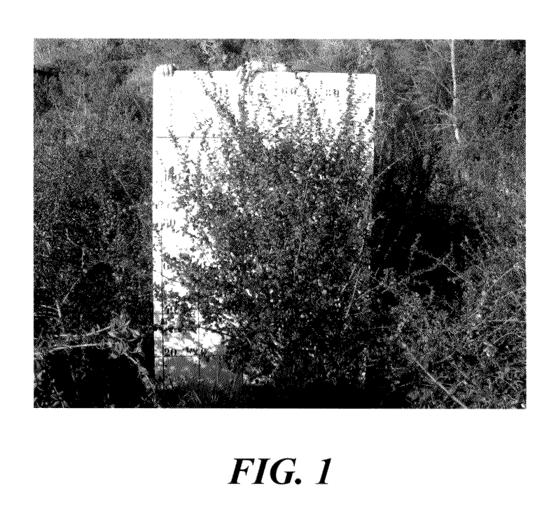

FIG. 2 shows the fruit of the ‘Red Pearl—INIA’ murtilla plant.

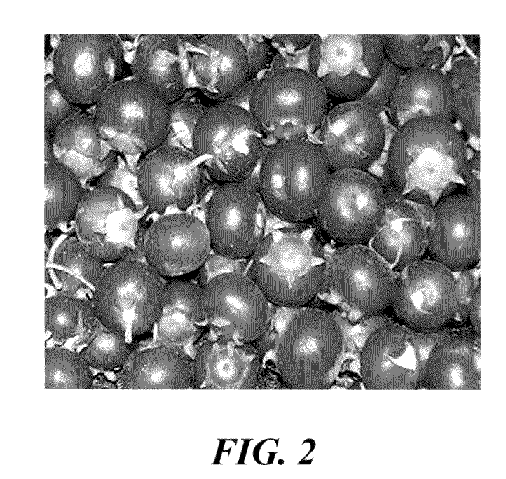

The colors of this illustration may vary with lighting conditions and, therefore, color characteristics of this new variety should be determined with reference to the observations described herein, rather than from these illustrations alone.

### DETAILED BOTANICAL DESCRIPTION

The following detailed botanical description is primarily based on observations of three year old plants made during the 2005 growing season at the INIA Carillanca Research Center at Temuco, Chile. It should be understood that the characteristics described will vary somewhat depending upon cultural practices and climatic conditions, and can vary with location and season. Quantified measurements are expressed as an average of measurements taken from a number of individual plants of the new variety. The measurements of any individual plant, or any group of plants, of the new variety may vary from the stated average. Where color descriptions are provided, reference is made to the Munsell Color Chart.
Plant: Type—Evergreen shrub; Growth habit—Erect; Height—131 cm; Diameter—62 cm; Internode length—13 cm.
Leaves: Length to width ratio—1.3; Leaf shape—Deltoid.
Fruit: Quantity of seeds per fruit—25.5; Fruit shape—spherical; Diameter (avg.)—1.0 cm (varies between about 0.9 cm and 1.1 cm, depending upon water and fertilizer application); Fruit skin color—Red 5R 3/8; Fruit pulp color—white; Peduncle length—12 mm; Yield—1,142 g/plant.

Fruit analytical data:

| | |
|---|---|
| Humidity (%) | 76.23 |
| Protein N × 6.25 (%) | 1.08 |
| Ashes (%) | 0.83 |
| Etereus Extract (%) | 0.92 |
| Fiber (%) | 3.24 |
| E.N.N. (%) | 17.69 |
| Pectin (%) | 0.32 |
| Total Sugar (sucrose) (%) | 9.78 |
| Reductors Sugar (glucosa) (%) | 4.65 |
| pH | 3.19 |
| Avg. Soluble Solids (° Brix) | 16 |
| (varies by growing region) | |
| Avg. Titratable Acidity (%) | 2.2 |
| (varies by growing region) | |

Bloom dates (calculated from planting date): Start of blooming—104 days; Full Bloom—154 days; End of blooming—168 days.
Pest and disease resistance/susceptibility: None noted.

What is claimed is:

1. A new and distinct murtilla plant as shown and described herein.
